(19) United States
(12) Patent Application Publication
(10) Pub. No.: US 2006/0165614 A1
(43) Pub. Date: Jul. 27, 2006

(54) Palatable micro-capsules

(76) Inventors: Gordon Nelson, Cheshire (GB); Stephen C Duckham, Merseyside (GB); Andrew E Round, Cheshire (GB)

Correspondence Address: BOYLE FREDRICKSON NEWHOLM STEIN & GRATZ, S.C., 250 E. WISCONSIN AVENUE, SUITE 1030, MILWAUKEE, WI, 53202, US

(21) Appl. No.: 10/534,948

(22) PCT Filed: Nov. 14, 2003
(86) PCT No.: PCT/GB03/04941

§ 371 Date: Oct. 13, 2005

(30) Foreign Application Priority Data
Nov. 16, 2002 (GB) 02267961

(51) Int. Cl. A61K 8/96; A61K 9/50

(52) U.S. Cl. 424/50; 424/490

(57) ABSTRACT

A formulation comprising at least one active or at least one ingredient, and a plurality of micro-capsules formed from a plurality of micro-organisms and having at least one flavouring encapsulated and passively retained within said micro-capsules, said flavouring not being a natural constituent of said micro-organisms, said micro-capsules having: (a) an at least substantially intact cell wall; and (b) an intact cell membrane; wherein said at least one active or said at least one ingredient is foul-tasting and said at least one flavouring masks, disguises or neutralises the foul-taste of said at least one active or said at least one ingredient, preventing a patient to whom said formulation is orally administered from tasting said foul-tasting active, or causing the flavour of said foul-tasting at least one active or ingredient to be reduced such that it is more palatable. Also disclosed are methods of manufacturing said formulations.

This invention relates generally to microbial micro-capsule formulations which contain foul tasting and/or foul-smelling ingredients or active ingredients (actives), which have been rendered palatable through the modulation of flavour, odour, texture, colour, temperature and/or viscosity.

Patents FR 2179528, U.S. Pat. No. 4,001,480, EP 0085805, GB 2162147 and EP 0242135, all describe methods/processes for the encapsulation of small molecules including actives inside micro-organisms such as yeast or bacteria.

Oral administration of actives to a patient currently represents the most convenient, cost effective and preferred form of drug delivery. The human tongue contains over 9000 taste buds which distinguish salt, bitter, sour, sweet and umami tastes. It will be appreciated that a major requirement of an orally administered active that contacts the taste buds is that the dosage form must be palatable, since an unpalatable formulation increases the risk of a patient neglecting to take the active. Such non-compliance with the dosing regimen can delay or prevent the patient's recovery from the condition under treatment.

Many useful, effective actives have a bitter taste when dissolved in liquid form or even when administered as pills or tablets. For example, a single Ciprofloxacin hydrochloride particle dissolving in the mouth has such a bitter, unpleasant taste that the product is invariably rejected by the patient. In the case of those compounds which have unpleasant (e.g. bitter) taste and/or odour characteristics, the provision of a dosage form represents a considerable problem. With improvements in flavour technology, patients now expect and demand orally administered medications that are pleasantly, or at least tolerably, flavoured. This is especially true with children and older adults for whom solutions are most often prescribed. Consequently, the palatability of orally administered actives is a major concern in the pharmaceutical industry.

Palatability of an orally administered active is influenced by a combination of sensory perceptions including taste and smell, and to a lesser extent, texture, appearance, and temperature of the formulation. In the case of microbial micro-capsules, although the micro-organism is usually intact or substantially intact after encapsulation of the active, in formulations where microcapsules are administered orally and the encapsulated active is foul-tasting, patients will detect the undesirable and unpleasant taste of the active within the microcapsules.

The present inventors have sought to provide formulations for oral administration wherein any unpleasant taste and/or odour characteristics of the active or ingredients are masked, disguised or neutralised through the use of an encapsulated flavouring, or combination of flavourings, which helps ensure quantitative intake of the intended dose, thereby reducing patient non-compliance.

According to a first aspect of the present invention there is provided a formulation comprising at least one active or at least one ingredient, and a plurality of micro-capsules formed from a plurality of micro-organisms and having at least one flavouring encapsulated and passively retained within said micro-capsules, said flavouring not being a natural constituent of said micro-organisms, said micro-capsules having:

(a) an at least substantially intact cell wall; and

(b) an intact cell membrane;

wherein said at least one active or said at least one ingredient is foul-tasting and said at least one flavouring masks, disguises or neutralises the foul-taste of said at least one active or said at least one ingredient, preventing a patient to whom said formulation is orally administered from tasting said foul-tasting active, or causing the flavour of said foul-tasting at least one active or ingredient to be reduced such that it is more palatable.

The term “active” as used herein is meant to include any drug, or therapeutic or otherwise active agent, preferably a pharmaceutical compound or chemical that is capable of being orally administered. Illustrative categories and specific examples of actives useful in conjunction with the present invention include: anti-viral agents, analgesics, anaesthetics, anorexics, anti-arthritics, anti-depressants, anti-diabetic agents, anti-inflammatory agents, anti-helminthics, anti-parkinsonism drugs, anti-pruritics, cardiovascular drugs, anti-hypertensives, ACE inhibitors, hormones, immunosuppressives, muscle relaxants, parasympatholytics, parasympathomimetics, psychostimulants, anti-tuberculosis agents, anti-tussives, such as dextromethorphan, dextromethorphan hydrobromide, noscapine, carbetapentane citrate, and chlophedianol hydrochloride; histamine H1-receptor antagonists, such as chlorpheniramine maleate, phenindamine tarrate, pyrilamine maleate, doxylamine succinate and phenyltoloxamine citrate; histamine H2-receptor antagonists, such as ranitidine, famotidine, cimetidine, nizatidine and roxatidine; decongestants, such as phenylephrine hydrochloride, phenylpropanolamine hydrochloride, pseudoephedrine, hydrochloride ephedrine; various alkaloids, such as codeine phosphate, codeine sulphate and morphine; mineral supplements such as potassium chloride and calcium carbonates, magnesium oxide and other alkali metal and alkaline earth metal salts; laxatives, vitamins; antacids; ion exchange resins such as cholestyramine; anti-cholesterolemic and anti-lipidic agents such as gemfibrozil; anti-arrhythmics such as N-acetyl-procainamide; anti-pyretics such as acetominophen, aspirin; non steroidic anti-inflammatory (NSAI) substances, and more particularly arylcarboxylic derivatives such as ibuprofen, ketoprofen, flurbiprofen, diclofenac, etodolac and naxoprene; NSAI oxicam derivatives such as piroxicam, meloxicam, tenoxicam, NSAI fenarnate, indolic, and phenylbutazone derivatives; appetite suppressants such as phenylpropanolamine hydrochloride or caffeine; and expectorants such as guaifenesin. Additional useful active medicaments include coronary dilators, cerebral dilators, peripheral vasodilators, anti-infectives, psychotropics, anti-manics, stimulants, gastrointestinal sedatives and bandages, anti-diarrhoeal and anti-constipation preparations, anti-anginal drugs, vasodilators, anti-hypertensive drugs, vasoconstrictors and migraine treatments, antibiotics, tranquillisers, anti-psychotics, anti-tumour drugs, anti-coagulants, and anti-thrombotic drugs, hypnotics, sedatives, anti-emetics, anti-nauseants, anti-convulsants, neuromuscular drugs, hyper- and hypoglycaemic agents, thyroid and anti-thyroid preparations, diuretics, anti-spasmodics, uterine relaxants, nutritional additives, anti-obesity drugs, anabolic drugs, erythiropoietic drugs, anti-asthmatics, anti-histaminic or anti-cholinergic or opiate derivatives (such as codeine, dextromethorphan, ethylmorphine, noscapine, pholcodine), cough suppressants, oral mucolytics (such as acetylcisteine, ambroxol, bromhexine, carbocisteine, erdosteine, letosteine), anti-uricemic drugs and the like. Other examples of actives are well known to a person skilled in the art.

The term “ingredient” is intended to include vitamins, nutritional supplements and nutriceutical products.

In this specification, the term “flavouring” generally refers to any substance used to improve, enhance, disguise, or mask the taste or odour of an active or ingredient contained within a formulation.

The term “foul-tasting” generally refers to an unpleasant taste and/or odour of an active or ingredient, as perceived by a patient to whom the active or ingredient is administered. The foul-taste may be due to bitter and/or salty and/or sour characteristics of the active or ingredient.

Microbial micro-capsules can be formulated for oral administration to a patient in a variety of ways, for example as a mouthwash, toothpaste, solution, suspension, gel, paste, powder, aerosol, tablet, chewable tablet, capsule, spray, lozenge, syrup, chewing gum, boiled sweet, or compressed sweet. The use of different formulations are well known to a person skilled in the art (Remigton's Pharmaceutical Sciences and US Pharmacopoeia, 1984, Mack Publishing Company, Easton, Pa., USA; United States Pharmacopoeia, ISBN: 1889788031). For example, in the case of a tablet, the micro-capsules containing encapsulated flavour may be used to coat the tablet, so that the micro-capsules contact the saliva and mucous membranes of the mouth, rather than the foul-tasting active.

The release of flavouring from microbial micro-capsules (yeast, fungi, bacteria, protozoa, and other unicellular organisms, including microbial derived materials which retain the cell wall structure such as that described in patent EP 0553176) can occur without physical breakage of the cell wall or chemical or biological degradation of the cell wall. Indeed, flavouring is released in a burst of activity when the micro-capsules are placed on a biological membrane such as the membrane coating the tongue. The burst of flavour activity associated with the micro-capsules contacting e.g. the tongue means that the flavour the patient experiences is an overwhelming taste of the flavouring rather than the foul-tasting active or ingredient. The foul-taste of the active or ingredient is masked, disguised or neutralised, typically completely, by the flavouring, and is not merely diluted by the flavouring. In the case of yeast micro-capsules for example, the yeast agglomerates (for example a spray dried agglomerated particle of an average diameter 30 microns) may contain a few hundred cells—these agglomerates readily disperse in the mouth when in contact with saliva down to individual cells and multiples of two or three cells allowing speedy contact and release of flavour, overcoming the slower reacting foul-tasting ingredient or active.

Preferably the flavouring is lipophilic—i.e. it is soluble within the lipid membrane of the micro-organism used for encapsulation.

Micro-capsules may be formulated wherein the at least one active or at least one ingredient is encapsulated and passively retained within the micro-capsules, the at least one active or the at least one ingredient not being a natural constituent of the micro-organisms.

Micro-capsules may be formulated with an at least one additional flavouring to further mask foul-taste and improve the palatability of the formulation. For example, in the case of micro-capsules (containing an encapsulated flavouring such as orange oil) formulated into a gelatin, glycerine, or glycerol mono-stearate capsule, the additional flavouring may be incorporated into the gel matrix of the capsule shell. The additional flavouring may also be e.g. orange oil. The use of an encapsulated flavour combined with an additional flavour is termed ‘dual masking’, and this technique improves taste and acceptability during and after swallowing, when a rebound aftertaste may occur. Such ‘dual masking’ is applicable in a wide range of preparations which contain strong tasting ingredients, such as fish oil, or garlic oil/powder, thereby eliminating the need for a deodorising process.

The flavouring may prevent a patient to whom the formulation is orally administered from tasting said foul-tasting ingredient or active, or the flavouring may reduce or lessen the foul taste associated with the ingredient or active, making the administered formulation more palatable.

There is often a correlation between the chemical structure of an active and its taste. Low molecular weight salts tend to taste salty where higher molecular weight salts tend toward bitterness. Nitrogen containing compounds, such as the alkaloids, tend to be quite bitter. Organic compounds containing hydroxyl groups tend to become increasingly sweet as the number of hydroxyl groups increase. Organic esters, alcohols, and aldehydes are known for their pleasant taste and cool sensation produced by their volatility. The determination of a foul-taste of an active is carried out by standard, well-known practices, and is a characteristic often listed along with a description of the active in texts such as The Merck Index, 11th ed., S. Budavari et al. eds., Merck & Co., Inc., Rahway, N.J. (1989) and Rernington's Pharmaceutical Sciences, 18th ed., A. Gennaro ed., Mack Publishing Co., Easton, Pa. (1990).

Examples of bitter and/or unpleasant tasting actives applicable to taste-masking are: Histamine H₂-antagonists, such as, for example, cimetidine, ranitidine, famotidine, nizatidine, etinidine, lupitidine, nifentidine, niperotidine, roxatidine, sulfotidine, tuvatidine and zaltidine; Antibiotics, such as penicillin, ampicillin, chloramphenicol, erythromycin, ciprofloxacin, norfloxacin, roxithromycin, cloxacillin and clarithromycin; Antimalarials such as chloroquin phosphate and quinine sulphate; Decongestants such as pseudoephedrine hydrochloride; Prokinetics such as metoclopramide hydrochloride; Antihistamines such as diphenhydramine, terfenadine, phenothiazine and chlorpheniramine; Nonsteriodal anti-inflammatory drugs (NSAIDs) such as ibuprofen, acetaminophen, nabumetone and naprosyn; Cough suppressants such as dextromethorphan hydrobromide. Other bitter and unpleasant tasting drugs include, caffeine, theophylline (asthma), spironolactone (aldosterone antagonist), guaifenesin (expectorant), prednisolone (corticosteroid), methacholine (bronchial challenge drug), neostigmine (acetylcholinesterase inhibitor), epinephrine (sympathomimetic), albuterol (Antiasthmatic, broncodilator; antihypertensive), chlorpromazine (sedative), chlordiazepoxide (librium—sedative, hypnotic, anxiolytic and muscle relaxant), amitriptyline (antidepressant), barbiturates, diphenylhydantoin (anticonvulsant), morphine (narcotic analgesic), meperidine (narcotic analgesic), lomotil (anti-diarrhoea), lidocaine (local anaesthetic). The above actives are not limiting and other foul-tasting actives will be well known to a person skilled in the art.

The flavouring may be one or more flavouring oils. For the purpose of this invention, flavouring oils used herein refer to both entire essential oils and the aroma chemicals malting up the essential oils. Essential oils are predominately volatile materials from botanical sources. The most widely used process for the isolation of essential oils is steam distillation of plant matter, although dry distillation and solvent extraction are also used. Essential oils are generally recognized as safe compositions that can be included in ingested materials. Aroma chemicals refer to chemicals which may be synthetic or natural, derived from essential oils, i.e., derived from plants by distillation, expression, or extraction, and which usually carry the flavour of the plant from which they are derived.

Although the invention is not limited to the specific essential oils listed individually in this specification, a number of important essential oils include: Almond-Bitter oil, Anise oil, Anise Star Dark oil, Gurjun Balsam oil, White Gurjun Balsam, Basil oil, Bergamot oil, Camphor oil, Caraway oil, Cassia oil, Cananga oil, Chamomile oil, Cherry oil, Cinnamon oil, Citronella oil, Clove Stern oil, Clove Leaf oil, Clove Bud oil, Cognac oil, Coriander oil, Cubeb oil, Eucalyptus oil, Eugenol oil, Ginger oil, Grapefruit oil, Jasmine oil, Laurel oil, Lavender oil, Lemon oil, Lime oil, Mace oil, Mandarin oil, Mayonara oil, Menthol oil, Mint oil, Nutmeg oil, Orange oil, Patchouli oil, Peppermint Yakima oil, Peppermint oil, Rose oil, Sage oil, Sassafras oil, Spearmint oil, Tangerine oil, Thyme oil, Violet oil, Vetiver oil, or Wintergreen oil.

Aroma chemicals include but are not limited to anethole, carvone, cintronellal, and camphor.

The flavouring may be one or more natural essences, for example an essence derived from Coffee, Tea, Chamomile, Cocoa, Ginger, Grape, Hazelnut, or Guava.

The flavouring may be one or more natural extracts. For example, the flavouring may be Almond Extract, Anise Extract, Caraway Extract, Cardamom Extract, Celery Seed Extract, Chocolate Extract, Cinnamon Extract, Clove Extract, Coriander Extract, Dark Cocoa Extract, Grand Marnier Extract, Lemon Extract, Lemon Lime Extract, Lime Extract, Mandarin Mint Extract, Orange Blossom Extract, Orange Extract, Parsley Herb Extract, Rum Extract, Tangerine Extract, Tarragon Extract, or Vanilla Extract Bourbon.

The flavouring may be one or more artificial flavourings, or natural flavourings.

For veterinary applications, the flavouring may be a savoury flavour which would be appealing to livestock and domestic animals such as dogs and cats. For example, the flavouring may be beef, roast beef, pork, ham, chicken, fish, crab, lobster, shrimp, or scallops.

The flavouring may be a spicy flavour, such as cinnamon, clove, jalapeno pepper, mace, or nutmeg.

The flavouring may be a nutty flavour. The flavouring may be almond, butter pecan, cashew, coconut, English walnut-black, hazelnut, peanut, pecan, pistachio, walnut, and walnut-black.

Alternatively, the flavouring may be one or more flavourings widely used in the pharmaceutical industry. Typically, such pharmaceutical flavourings have long lasting taste profiles and well characterised taste masking properties. Pharmaceutical flavourings include: Anise, Apple, Apricot, Banana, Blackberry, Blueberry, Brandy, Butter, Butter Rum, Butterscotch, Caramel, Champagne, Cherry-Black, Cherry-Maraschino, Cherry-Red, Cherry-Wild, Cherry Apricot, Cherry Mint, Coconut, Coffee, Cognac, Cola, Cranberry, Cream Soda, Currant-Black, Egg Nog, Fennel, Ginger Ale, Grape, Grapefruit, Grenadine, Hazelnut, Lemon, Lemon-Lime, Maple, Maple Walnut, Mint Orange, Passion Fruit, Peach, Pineapple, Plum, Prune, Raspberry, Root Beer, Rum, Rum & Coffee, Sherry, Spearmint, Tangerine, Tutti Frutti, or Vanilla Custard.

Flavourings such as aroma chemicals, natural essences, essential oils, natural extracts, artificial flavours, natural flavours and pharmaceutical flavours are commonly available, for example from Blue Pacific Flavours & Fragrances, Inc., (1354 South Marion Court, City of Industry, Calif., USA 91745-2418, www.bluepacificflavors.com).

The flavouring may be a sweetener. Sweeteners can be both natural and synthetic. In this specification, the term “sweetener” refers to sweet substances which are conventionally known or will possibly be known in the future. Examples of the sweet substances include sucralose, alpha-glucosyltransferase-treated stevia, alpha-cyclodextrin, beta-cyclodextrin, aspartame, acesulfame potassium, N-acetylglucosamine, arabinose, alitame, isotrehalose, isomaltitol, isomaltooligosaccharide (isomaltose, isomaltotriose, panose, etc.), erythritol, oligo-N-acetylglucosamine, galactose, galactosylsucrose, galactosyllactose, galactopyranosyl (beta 1-3) galactopyranosyl (beta 14) glucopyranose, galactopyranosyl (beta 1-3) glucopyranose, galactopyranosyl (beta 1-6) galactopyranosyl (beta 1-4) glucopyranose, galactopyranosyl (beta 1-6) glucopyranose, glycyrrhiza extract (glycyrrhizin), xylitol, xylose, xylooligosaccharide (xylotriose, xylobiose, etc.), glycerol, triammonium glycyrrhizinate, tripotassium glycyrrhizinate, trisodium glycyrrhizinate, diammonium glycyrrhizinate, dipotassium glycyrrhizinate, disodium glycyrrhizinate, curcurin, glucose, gentiooligosaccharide (gentiobiose, gentiotriose, gentiotetraose, etc.), saccharin, sodium saccharin, cyclamate, sucrose, stachyose, stevia extract, powdered stevia, dulcin, sorbitol, sorbose, thaumatin, Theander oligo saccharide, tenryocha extract, trehalulose, trehalose, monellin, nigerooligosaccharide (nigerose, etc.), neotame, neotrehalose, palatinit, palatinose, palatinosepalatinose oligosaccharide, palatinose syrup, fucose, fructooligosaccharide (kestose, nystose, etc.), fructosyl transferase-treated stevia, fructofuranosyl nystose, Brazilian licorice extract, fructose, polydextrose, maltitol, maltose, maltosyl beta-cyclodextrin, maltotetraitol, maltotriitol, maltooligosaccharide (maltotriose, tetraose, pentaose, hexaose, heptaose, etc.), mannitol, miracle fruit extract, melibiose, rakanka (Momordica grosvenori)) extract, lactitol, lactulose, lactose, raffinose, rhamnose, ribose, isomerized corn syrup, reduced isomaltooligosaccharide, reduced xylo-oligosaccharide, reduced gentiooligosaccharide, reduced malt sugar syrup, glucose syrup, hydrogenated glucose syrup, enzymatically modified licorice, licorice hydrolysates, coupling sugar, soybean oligosaccharide, inverted sugar, glucose syrup, honey and like sweet substances.

The amount of the flavouring to be used in oral formulations of microbial microcapsules in accordance with this invention may be fixed at any desired (e.g. a conventional) level. Typical flavour loading may be for example 0.5-2% w/w Peppermint oil, 0.5-1% w/w Lemon oil, 0.5%-1% w/w Strawberry flavour (Ungurer), 0.5-2% Aniseed oil. All flavours are typically encapsulated to the maximum possible level of encapsulation within a 4-hour incubation. Actives may be diluted with maltodextrin to a standard 20% W/w flavour loading.

If the encapsulated active is particularly foul tasting (e.g. extremely bitter), the addition of a sweetener such as e.g. aspartame may only partially mask or disguise the taste. A further debittering agent, such as ammonium glycyrrhizinate may be used to more fully mask or disguise the taste of the active. Ammonium glycyrrhizinate may be used at a weight ratio to the otherwise bitter-tasting active of about 1:50 to about 1:10, and most preferably at weight ratio of about 1:20 ammonium glycyrrhizinate to active. Ammonium glycyrrhizinate is the mono-ammonium salt of a triterpenoid saponin that consists of an aglycone of glycyrrhetic acid and a sugar moiety of two glucuronic acid units linked to each other. This material is about 50 to about 100 times sweeter than sucrose, and is known to be useful in masking bitterness.

The bitterness-masking properties of ammonium glycyrrhizinate may further be enhanced through the use of Polyvinylpyrrolidone (PVP)— these compounds appear to potentiate each other to provide the desired bitterness-masking effect. For example, a particularly bitter-tasting active such as Ciprofloxacin hydrochloride may be formulated with microbial microcapsules containing PVP (5-30% by weight), ammonium glycyrrhizinate (0.01-0.5% by weight), together with one or more flavourings. Alternatively, the micro-capsules containing flavouring may be used to coat a tablet for example, which comprises the foul-tasting active, together with PVP and ammonium glycyrrhizinate.

Discovering the flavouring best suited to masking an unpleasant taste is often a very empirical matter. However, general guidelines regarding the type of flavour best suited to mask a given taste are well known to a person skilled in the art. For example, salty tastes may be masked through the use of cinnamon, raspberry, orange, maple, and butterscotch flavours, bitter tastes may be masked through the use of cocoa, chocolate-mint, wild cherry, walnut, and raspberry flavours, and sour tastes may be masked through the use of fruit, citrus, and cherry flavours.

The age of the patient and the frequency of dosing will further influence patient taste preferences. Children generally prefer sweet, fruity, and candy-like tastes while adults prefer less sweet, tart-fruity flavours. Flavouring agents are generally unnecessary and not recommended for infants under 3-6 months of age. Older adults may prefer mint or even wine flavoured vehicles. Since liquids which require frequent administration, such as antacids, may rapidly become tiresome with a sweet fruity taste, they may be formulated in mint or tart citrus flavours.

Although altering taste perception by masking unpleasant tastes with a flavouring agent is the major factor in producing palatable formulations containing microbially encapsulated flavourings, other factors contribute as well. Odour is a strong determinant of taste perception, and the aroma or scent of any oral solution should be pleasant and should correlate with the flavour. Since product temperature can also influence taste perception, refrigerating liquid products can generally reduce unpleasant taste. Product texture also plays a role in taste perception and patient acceptance. Gritty or chalky preparations are poorly received by most patients (as attested to by the large amounts of money spent by antacid manufacturers to promote their non-chalky, non-gritty products).

The role of colour can also be important in determining patient acceptability for a formulation. Clear, water-like solutions may be poorly accepted on the basis of perceived inertness or lack of potency. Dark colours, often associated with poisons, such as dark purple, navy, black and brown, may also be rejected. More pleasant, fruity colours are generally preferred and should be coordinated with flavours and scents (e.g., yellow-lemon, red-cherry). In formulations where masking of the foul taste of an ingredient or active is only partially possible, an idiosyncratic selection of flavouring, colouring and odour may distract the patient sufficiently long enough to allow the formulation to be administered. For example, a particularly bitter active can be partially masked in a blue coloured, strawberry flavoured, mint-scented formulation. The theory is that while the brain reconciles the contradictions of a minty, blue strawberry, the foul-tasting ingredient or active has been swallowed.

Texture should also be considered in the preparation of any orally administered formulation. Viscosity plays an important role in patient acceptance—the characteristic viscosity of syrups appears to have a positive effect on patient acceptance, whereas less viscous solutions may be perceived as “watered down” and more viscous solutions as unpleasant. In the formulation of microbial micro-capsules as a suspension it is important to ensure that the particle size is sufficiently small enough so that the product is non-chalky or non-gritty and that the viscosity is such that the product is fluid enough to flow while being viscous enough to maintain particle dispersion.

According to a second aspect of the present invention, there is provided the use of an at least one flavouring in the manufacture of a formulation, said formulation comprising at least one active or at least one ingredient, and a plurality of micro-capsules formed from a plurality of micro-organisms, said micro-capsules having:

(a) an at least substantially intact cell wall; and

(b) an intact cell membrane;

said at least one flavouring being encapsulated and passively retained within said micro-capsules, said flavouring not being a natural constituent of said microorganisms, wherein said at least one active or said at least one ingredient is foul-tasting and said at least one flavouring masks, disguises or neutralises the foul-taste of said at least one active or said at least one ingredient, preventing a patient to whom said formulation is orally administered from tasting said foul-tasting active, or causing the flavour of said foul-tasting at least one active or ingredient to be reduced such that it is more palatable.

According to a third aspect of the present invention, there is provided a method of manufacture of a formulation, said formulation comprising at least one active or at least one ingredient, and a plurality of micro-capsules formed from a plurality of micro-organisms and having at least one flavouring encapsulated and passively retained within said micro-capsules, said micro-capsules having:

(a) an at least substantially intact cell wall; and

(b) an intact cell membrane;

said flavouring not being a natural constituent of said micro-organisms, wherein said at least one active or said at least one ingredient is foul-tasting and said at least one flavouring masks, disguises or neutralises the foul-taste of said at least one active or said at least one ingredient, preventing a patient to whom said formulation is orally administered from tasting said foul-tasting active, or causing the flavour of said foul-tasting at least one active or ingredient to be reduced such that it is more palatable, comprising the steps of:

(i) contacting said micro-organisms with said at least one flavouring, said at least one flavouring being capable of diffusing into the cell wall of said micro-organism without causing total lysation thereof, whereby said at least one flavouring is absorbed by said micro-organisms by diffusion across the cell wall and is retained passively within said micro-organism to produce a plurality of micro-capsules containing said at least one flavouring,

(ii) formulating said micro-capsules with said at least one active or ingredient.

The flavouring may be encapsulated according to any of the methods described in Patents FR 2179528, U.S. Pat. No. 4,001,480, EP 0085805, GB 2162147 and EP 0242135. These encapsulation methods are not limiting and other methods may be known to a person skilled in the art.

The micro-organism is preferably a fungus. Typical fungi are yeasts e.g. Saccharomyces cerevisiae (brewer's yeast and baker's yeast), Kluyveromyces fragilis (dairy yeast), Saccharomyces bayanus (wine yeast) and Candida utilis. Yeasts may be selected from the taxonomic order Endomycetales. The micro-organism may be a filamentous fungus, e.g. Aspergillus niger. The spore, mycelium and giant cell forms of filamentous fungi may be employed. The micro-organism may be a mold, e.g. Fusarium graminearium. Other micro-organisms which may be employed are bacteria and algae. Any relatively large protozoa also may be utilised—examples of such protozoa include Bacteriodes succinogenes, Etidinium ecaudatum, Entodinium caudatum, Eudipolodinium neglectum, Eudiplodinium maggii, Diplodinium dentatum, and Polyplastron multivesiculatum.

The flavouring should be in liquid form during the encapsulation process and should be soluble in the cell membrane for encapsulation to take place. The flavouring may be a liquid (including oil) in its normal state, or it may be a solid, in which case it should be dissolved or micro-dispersed in a solvent which is lipid soluble. Suitable solvents include:

(a) primary alcohols within the range C4 to C12, such as nonanol and decanol (higher alcohols containing a linear chain of more than twelve carbon atoms are too large for encapsulation);

(b) secondary and tertiary alcohols;

(c) glycols such as diethylene glycol;

(d) esters—any ester where the straight carbon chain is greater than 2 and less than or equal to 12, e.g ethyl butyrate, triacetin;

(e) aromatic hydrocarbons such as xylene, and acetophenone,

(f) any aromatic lipophilic oil with no straight chain branch greater than 12 carbons.

(g) carboxylic acids between C3 and C12.

Solid flavourings may be encapsulated, however it should be lipophilic to encapsulate successfully and it should either melt below 80° C. or be soluble in one of the above solvents. Prolonged temperatures above 80° C. would damage the cell membrane beyond repair. Ideally for the process the flavouring should be liquid between 40 and 65° C. since higher temperatures may result in degradation of the flavouring.

Multiple flavourings may be co-encapsulated—e.g. a combination of essential oils.

In instances where the flavouring is an oil, such as peppermint oil, a solid active, ingredient or additional flavouring can be dissolved in the oil and co-encapsulated with it. The oil serves as a solvent for the active, ingredient or additional flavouring, and also imparts flavour to the resulting micro-capsules. The suitability of flavourings for encapsulation may be found by a simple trial of the method of the invention.

Methanol, ethanol and isopropanol are very low molecular weight volatile solvents, which can be used to assist in encapsulation but do not actually encapsulate themselves. If used to encapsulate a material the flavouring must be soluble in e.g. ethanol and when added to e.g. 3 or 4 parts water the active must stay in solution. There must always be some water present to swell the yeast thereby hydrating the membrane, or encapsulation will not take place. The ethanol evaporates during the process and the flavouring, which must be at least partially soluble within the yeast membrane, is encapsulated. Residual ethanol will evaporate during post-encapsulation treatments such as spray drying.

Several criteria must be considered in order to predict whether a flavouring can be encapsulated. Flavourings having a benzene or naphthalene ring appear to be particularly suitable for encapsulation. Actives with an octanol/water (log P) partition coefficient of between 0.5 and 4.0 will encapsulate well. Molecular weight must also be considered—flavourings with a molecular mass less than 1000 Daltons can generally be encapsulated. Size is also important—since straight chain hydrocarbons greater than C12 generally do not encapsulate, any molecule containing a straight chain C12 stretch or greater will probably not encapsulate, nor will a molecule with a rigid structure similar in length to a C12 chain. Molecules with a greater number of carbons than C12 can be encapsulated as long as the structure contains benzene rings, e.g. phenolics, or naphthalene rings, etc. Molecules with a small molecular diameter are typically encapsulated most efficiently. Volatile molecules with one to three carbons generally do not encapsulate, e.g. ethane, ethanol, propanol, whereas molecules containing four or more carbon atoms generally do encapsulate. The optimal range for encapsulation in terms of straight chain carbon atoms lies between C4-C12. Beyond these criteria, the suitability of flavourings for encapsulation may be found by a simple trial of the method of the invention.

The encapsulation treatment may be performed at normal ambient temperatures but preferably the temperature is elevated, in order to expedite the encapsulation treatment. A suitable elevated temperature may be in the range 35 to 60° C., for instance in the range 40 to 50° C.

The encapsulation treatment preferably comprises mixing the micro-organism with the flavouring in a liquid medium, especially an aqueous medium, to attain good dispersion and contact of the micro-organism with the flavouring.

The encapsulation treatment may be continued until optimum encapsulation has been achieved. Encapsulation may usually be observed microscopically as one or more globules of the flavouring contained within the microbial cell, unless the yeast is grown in a harsh environment (such as high alcohol content), in which case the cell wall can be thickened which makes direct visualisation by light microscopy more difficult. In such instances, transmission electron microscopy (TEM) may be required. The encapsulation treatment may take a few hours before the optimum level of encapsulation is achieved.

After encapsulation, residual low molecular weight solvents such as ethanol, methanol and propanol may be removed after the encapsulation process through processes such as spray drying. Water may also be removed by spray- or freeze-drying. Water may also be removed by evaporation by putting the micro-capsule suspension in a dry oven.

A pre-treatment bleaching step may be carried out prior to encapsulation. For example, the treatment may be performed at room temperature for up to one hour where the micro-organism is treated with a solution of an alkaline bleach solution comprising 0.2 M sodium hydroxide/1% w/v hydrogen peroxide, with a pH value of between 9-10. Sodium silicate may be added to the mixture as an anti-foam agent. The resulting micro-organisms are generally off-white in colour, and the cell wall may be more porous. For example, in the case of bleached yeast, the cells when dry may absorb between 5-10 times their weight in water, compared to untreated yeast cells which may absorb between 2-3 times their weight in water. This increased capacity of the bleached yeast to absorb water means that encapsulation is usually performed in a greater volume of liquid, thereby avoiding problems associated with increased viscosity.

Prior to, or in some cases during, the encapsulation process, the micro-organism may be treated at an elevated temperature and/or with an enzyme and/or with a chemical such as a magnesium salt to improve the efficiency of encapsulation. Enzymes such as pepsin, trypsin, chymotrypsin, chitinase, and β-glucanase serve to degrade the microbial cell wall. Magnesium salts enhance permeability of the micro-organism. The micro-organism may then be mixed with the flavouring to be encapsulated and incubated until optimum encapsulation is achieved (as determined by light or electron microscopic analysis of the micro-capsules).

After encapsulation of the flavouring, and prior to preparation of the formulation, a conditioning treatment of the resulting micro-capsules may be performed to remove any residual microbial taste, colour and odour of the micro-capsules. This conditioning treatment may comprise incubating the micro-capsules in a dry environment such as an oven or heat chamber at room temperature for several weeks or months, or at an elevated temperature of up to 40° C. for hours/days.

In the case of yeast, the encapsulation process results in the accumulation of actives within the naturally double walled capsule. Yeast cell walls are generally 80-90% polysaccharide, including predominant glucans such as 1,3-β-glucan, and also the long chain carbohydrate polymer chitin which adds rigidity and structural support to the cells. Proteins (such as mannoproteins), lipids and polyphosphates together with inorganic ions make up the cell wall cementing matrix. The inner membrane is a typical lipid bilayer. The yeast cell wall, unlike many food grade capsules, is insoluble and therefore the micro-capsules can be wet and dry processed. When the yeast microcapsules are spray dried a free flowing powder is produced made up of agglomerated particles comprising numerous yeast cells. Depending on drying conditions the dry particle size can range between 10 and 300 microns. For large particles a fluidised bed is required. The product can also be prepared as a cake, suspension, produced by pressing, or rotary drying. Particle size or a mixture of particle sizes may be useful to control release rates.

The micro-capsules may be washed after encapsulation to remove residual unencapsulated material and isolated by centrifuging, freeze-drying or spray-drying.

The invention will be further apparent from the following experiments which show, by way of example only, formulations of the present invention and methods of manufacture of same.

### EXPERIMENTS

The following examples detail various formulations of actives and flavourings for oral administration, and the methods used to manufacture such formulations.

### Example 1

Saccharomyces cerevisiae 62F was sourced from Williams Bio-Energy, Pekin Ill. The spray dried yeast was pre-washed with water to remove residual media components and the resulting washed yeast suspended in water to a final concentration of 33% w/v. Peppermint oil was added to a final concentration equivalent to 50% of the dry weight of the yeast. The mixture was stirred at 45° C. for 6 hours in a water-jacketed vessel using a Teflon coated paddle. The yeast cells containing encapsulated peppermint oil were harvested by centrifugation, 2000×g, and washed with water twice to remove residual unencapsulated flavour. The washed yeast micro-capsules containing peppermint oil were diluted to 35% dry solids with water and spray dried. The resulting powder had an average particle size of 30 microns, each containing approximately 150-250 yeast cells each containing peppermint oil. The yeast encapsulated peppermint oil was found to enhance the perception in the mouth of the peppermint oil compared to unencapsulated free peppermint oil, by a factor of between 4 and 5. When flavour oil at a commonly used level in the pharmaceutical industry was added to Chloroquin sulphate, a bitter tasting anti-malarial compound, the Chloroquin sulphate could be easily detected when placed on the tongue. When an equivalent weight of yeast-encapsulated flavour was used the Chloroquin sulphate was no longer perceived.

### Example 2

Torula yeast (Candida utilis) obtained from Overseal Ltd., was washed with water to remove free dissolvable nutrients and extraneous material. The resulting washed yeast were adjusted with water to a final concentration of 30% dry solids and stirred using a rotary stirrer at 380 rpm. To this suspension, Ungurer strawberry flavour (a liquid preparation) was added to approximately 50% the dry weight of the dry weight of the yeast cells. With continuous stirring the mixture was incubated in a water jacketed, closed vessel for 3 hours at 55° C. After this time the yeast cells were harvested by centrifugation and freeze dried. When placed in the mouth the dry powder (20 mg) produced an intense strawberry flavour, 3-4 times that found with the equivalent free flavour. The flavour was able to mask Pseudoephedrine HCl, a bitter tasting decongestant compound mixed into the powder at up to 15% by weight.

### Example 3

Spray dried (400 g) brewers yeast (Saccharomyces cerevisiae NCYC 1335) was suspended in a mixture of 500 ml of saturated acesulfame K and 500 ml of saturated sucralose. The mixture was incubated at 30° C. for 1 hour and dried by freeze-drying. The resultant product was gently milled to a particle size of 100 microns. The product delivered an intense sweetening effect which was able to overcome the flavour of Metoclopramide, a bitter tasting prokinetic compound. Microscopic examination confirmed that not all the sweetener was encapsulated and a portion was located on the surface of the capsules.

The same process was carried out using Williams Bioenergy yeast 62F, which had been bleached using the method described in Example 7. In this case 1000 ml of acesulfame K and 1000 ml of sucralose was used because of the ability of the bleached yeast to take up greater volumes of water. The cell wall is also opened up more effectively allowing more space to be filled with the sucralose. As before on drying the product contained a mixture of encapsulated sweetener and unencapsulated sweetener.

### Example 4

As perception of flavour, particularly with children is often due to expectation based on the colour of the product, during encapsulation of lime oil in yeast, a food grade green dye was added to the mixture at 0.1% w/v. In this case 250 ml Lime oil (R. C Treate) was added to 1 litre of water containing 500 g of spray dried Williams Bio-Energy yeast 62F. The mixture was stirred for 4 hours at 50° C. When washed with Water and dried using spray drying the product contained 24% lime oil and was green in colour.

### Example 5

With domestic animals, (e.g. dogs and cats), although the flavours may be similar the format of delivery may be different. To make foul-tasting actives such as the anti-worming agent dichlorophen more palatable, yeast encapsulated beef flavour was added to the foul-tasting actives. In this case, a yeast was chosen which had an inherently beefy flavour, a distiller yeast sourced from Quest International Ltd. The yeast was dispersed in water to a final concentration of 32% w/v and the liquid flavour added to 50% the dry weight of the yeast. The mixture was incubated whilst stirring for 8 hours at 40° C. The yeast cells containing the beef flavour were harvested by centrifugation, washed with water and dried by spray drying. The yeast capsules contained approximately 15% flavour oil. For dogs, in which the product would not remain in the mouth for a long period and little chewing will take place, a thin coating of the yeast encapsulated intensely beefy flavoured product over a tablet of the foul tasting material was effective. For cats where more chewing behaviour takes place before ingestion, the intense yeast encapsulated beefy flavour was mixed with no greater than 20% of the foul tasting compound, again which was effective.

### Example 6

Saccharomyces cerevisiae NCYC 1603 was maintained on MYGP agar slopes (0.3% (w/v) each of malt extract and yeast extract, 0.5% bacterial peptone, 2% (w/v) glucose; 2% (w/v) agar). A loop of yeast was transferred aseptically to 10 ml MYGP broth, media as above but without agar and incubated overnight at 30° C. The broth was aseptically transferred to a fermenter containing 5-litres working volume of MYGP broth. The fermenter was incubated for 3 days at 30° C. and the yeast harvested by centrifugation using a MSE Mistral 3000i centrifuge (2000×g). The harvested yeast was washed with water to remove excess media and suspended in water to a final solids content of 33% w/v in a jacketed glass vessel, temperature 42° C. The yeast was agitated with top stirring, Stuart Scientific SS10, with Teflon paddle, at approximately 300 rpm. Peppermint oil (125 g) and spearmint oil (125 g) were added and the suspension continually stirred for 6 hours. The cells were harvested by centrifugation, 2000×g using a Mistral 3i Centrifuge for 20 minutes. The pelleted yeast cells containing encapsulated peppermint and spearmint oil was washed twice with water and the cells re-suspended in water to approximately 35% solids. The material was then spray-dried using a Niro Mobile Minor spray dryer. The peppermint oil and spearmint oil concentration was found to be 15% and 17%, respectively.

The dry cells were coated onto the surface of a preformed tablet containing Dextromorphan, a bitter tasting cough suppressant compound. When the surface layer came in contact with the mouth and saliva in combination, an intense flavour was released, greatly facilitating speedy swallowing of the tablet, with no bitter taste detected. A portion of the yeast capsules on the surface coating freely dispersed within the mouth, enhancing the taste-masking effect. The high intensity flavour release encouraged saliva production, easing swallowing of the tablet containing the active.

### Example 7

Commercially available dry bakers yeast (300 g) (Saccharomyces cerevisiae) sourced from DCL Ltd., was suspended in one litre of a 0.2 M solution of sodium hydroxide in water containing 40 g per litre of sodium silicate. Hydrogen peroxide was added until the concentration reached 1% w/v and the resulting suspension was then gently stirred for one hour at room temperature. The yeast was then removed by centrifugation, washed with water to remove excess bleaching agent and dried by spray drying. The yeast produced was white to off-white in colour and in suspension had a creamy texture. Any yeasty odour had been removed. The spray-dried material was stored dry at room temperature ready for future encapsulation processes.

A portion of the suspension before drying, was adjusted to 20% solids with water. The viscosity of the bleached and deodorised yeast was too great to obtain the desired emulsion characteristics using a similar concentration as the unbleached yeast. The bleached and deodorised yeast suspension was stirred using a rotary stirrer at 350 rpm for 5 hours at 40° C. in the presence of liquid caramel flavour to approximately 50% of the weight of dry yeast. The yeast cells containing liquid caramel flavour oil were then removed by centrifugation, washed with water and dried by spray-drying. The dry product contained 26% liquid caramel oil.

The dry capsules were mixed with a crystalline formulation of a bitter tasting active (Chloropheniramine maleate—an anti-histaminic), ensuring that the flavour was in excess of the bitter tasting active. For best results the yeast capsules containing flavour were in a ratio of 3:1 or greater with the bitter tasting active. The mixture of yeast encapsulated flavour and bitter tasting active were formed into a tablet using standard methods known in the pharmaceutical industry.

### Example 8

Saccharomyces bayanus (a wine yeast) was suspended in water to a final dry-solids concentration of 35% w/v. Caffeine was dissolved in peppermint oil, to 10% w/v, and mixed with the yeast whilst stirring at 350 rpm, to a final concentration of 50% of that of the dry yeast for 8 hours at 42° C. With this application care must be taken not use a solution of bitter tasting active of greater than 30% solubility in the peppermint or other flavour oil carrier. If the concentration of bitter tasting compound is greater there is a danger that the bitter taste would also be enhanced over the ability of the flavour oil to mask the foul tasting material. The yeast cells containing encapsulated peppermint oil/caffeine were harvested by centrifugation, and the cells were washed twice with water. The cells contained 28% w/w peppermint oil and 3.2% caffeine. After formulating the yeast containing peppermint oil and caffeine into a tablet, the product was rendered palatable.

### Example 9

Kluyveromyces lactis, (a commercially available yeast) cultured on whey was suspended in water to a final dry solids concentration of 33% w/v. The yeast suspension was stirred using a rotary stirrer at 350 rpm for 3 hours at 60° C. in the presence of menthol, which had been pre-melted, added to approximately 50% of the weight of dry yeast. The yeast cells containing menthol were then removed by centrifugation, washed with warm water and dried by spray-drying. The dry product contained 28% menthol crystals. When placed on the tongue there was a intense cooling/menthol flavour detected. Bitter tasting compounds e.g. Ciprofloxacin HCl (an antibacterial) could be added to the mixture to a level of 20% before overcoming the intense cooling/menthol flavour.

### Example 10

Saccharomyces cerevisiae NCYC 1338, (a non-flocculant yeast) was suspended in water to a final concentration of 35% dry solids. To this suspension, five times concentrated lime oil was added to 50% of the dry weight of yeast, and the mixture was agitated using a rotary stirrer at 300 rpm. The mixture was stirred for 2 hours at 60° C. in the presence of a nitrogen sparge, to reduce potential for oxidation. The cells were harvested by centrifugation at 2000×g and the residual lime oil was removed by washing with water. An aliquot of the concentrated pellet was re-suspended in a saturated solution (approx 30% w/v) of acesulfame-K (a sweetener) and incubated with gentle stirring (100 rpm) in a rotary stirrer for 30 minutes until the sweetener had diffused into the yeast cells. The preparation was then dried by spray drying, resulting in a powder with yeast cells containing 28% w/w lime oil and 300 g of acesulfame-K. On microscopic examination it was noted that not all the sweetener had entered the cell, so the powder included yeast capsules containing lime oil in the cell cytoplasm, sweetener in the cell wall matrix and free sweetener.

The second aliquot of the suspension yeast micro-capsules containing lime oil was dried by spray drying and re-suspended in a saturated solution of acesulfame-K. After incubation and drying the relative concentration of material entrapped within the yeast cell wall matrix was greater than when the yeast suspension was mixed with the sweetener solution.

Both of these products, when formulated with a bitter tasting active (Norflaxacin—an antibacterial), delivered intense flavour and strong sweetening effects thereby masking the foul-taste of the active.

### Example 11

Where the yeast used in the process would require to be registered as ‘Organic’, for example in nutriceutical applications, Bioreal yeast (Agrano GmbH) can be used. A sample of Bioreal yeast was suspended in water to a final dry solids concentration of 35% and essential orange oil was added to a final concentration equal to 45% of the dry weight of the yeast. The mixture was incubated whilst stirring for 6 hours at 48° C. After washing and spray-drying, the yeast cells contained 26% orange oil. The encapsulated flavour produced an intense orange flavour, 4-5 times that of the equivalent free oil. The yeast encapsulated orange oil was able to mask up to 20% addition of Cloxacillin sodium—a bitter tasting antibacterial compound.

### Example 12

Saccharomyces cerevisiae (62F) was obtained from William Bioenergy as a spray dried powder, this yeast was light in colour and had little yeast flavour due to the chosen culture media, which was based on corn syrup. The dry powder washed with water to remove excess media components and the resultant yeast, approximately 65% of the dry weight of the spray dried powder, was suspended in water to a final solids content of 35% w/v in a jacketed glass vessel, temperature 42° C. The yeast was agitated with top stirring, Stuart Scientific SS10, with Teflon paddle, at approximately 300 rpm. Ibuprofen dissolved in peppermint oil (10% w/v) was added to the mixture to approximately half the dry weight of the washed yeast and the mixture stirred continuously for 6 hours. The yeast cells containing peppermint and ibuprofen were then removed by centrifugation, washed with warm water and dried by spray-drying. The resulting yeast capsules contained 36% w/w peppermint oil and 3.7% w/w ibuprofen. The resulting powder was rendered palatable and the bitter taste of ibuprofen was not detectable.

We claim:

1. A formulation comprising at least one active or at least one ingredient, and a plurality of micro-capsules formed from a plurality of micro-organisms and having at least one flavouring encapsulated and passively retained within said micro-capsules, said flavouring not being a natural constituent of said micro-organisms, said micro-capsules having:
(a) an at least substantially intact cell wall; and
(b) an intact cell membrane;
wherein said at least one active or said at least one ingredient is foul-tasting and said at least one flavouring masks, disguises or neutralises the foul-taste of said at least one active or said at least one ingredient, preventing a patient to whom said formulation is orally administered from tasting said foul-tasting active, or causing the flavour of said foultasting at least one active or ingredient to be reduced such that it is more palatable.

2. A formulation according to claim 1, said at least one active or said at least one ingredient being encapsulated and passively retained within said micro-capsules, said at least one active or said at least one ingredient not being a natural constituent of said microorganisms.

3. A formulation according to claim 1, wherein said formulation additionally comprises an at least one additional flavouring.

4. A formulation according to claim 1, in which said at least one flavouring or said at least one additional flavouring is selected from the group consisting of: essential oils, aroma chemicals, natural essences, natural extracts, sweeteners, artificial flavourings, natural flavourings, pharmaceutical flavourings, or nature identical flavourings.

5. A formulation according to claim 1, wherein said at least one flavouring or said at least one additional flavouring additionally comprises ammonium glycyrrhizinate and/or polyvinylpyrrolidone.

6. A formulation according to claim 1, selected from the group consisting of: mouthwash, toothpaste, solution, suspension, gel, paste, powder, aerosol, tablet, chewable tablet, capsules, spray, lozenge, linctus, syrup, chewing gum, boiled sweet, or compressed sweet.

7. The use of an at least one flavouring in the manufacture of a formulation, said formulation comprising an at least one active or at least one ingredient, and a plurality of micro-capsules formed from a plurality of micro-organisms, said micro-capsules having:
(a) an at least substantially intact cell wall; and
(b) an intact cell membrane;
said at least one flavouring being encapsulated and passively retained within said microcapsules, said flavouring not being a natural constituent of said micro-organisms, wherein said at least one active or said at least one ingredient is foul-tasting and said at least one flavouring masks, disguises or neutralises the foul-taste of said at least one active or said at least one ingredient, preventing a patient to whom said formulation is orally administered from tasting said foul-tasting active, or causing the flavour of said foultasting at least one active or ingredient to be reduced such that it is more palatable.

8. A method of manufacture of a formulation, said formulation comprising at least one active or at least one ingredient, and a plurality of micro-capsules formed from a plurality of micro-organisms and having at least one flavouring encapsulated and passively retained within said micro-capsules, said micro-capsules having:
(a) an at least substantially intact cell wall; and
(b) an intact cell membrane;
said flavouring not being a natural constituent of said micro-organisms, wherein said at least one active or said at least one ingredient is foul-tasting and said at least one flavouring masks, disguises or neutralises the foul-taste of said at least one active or said at least one ingredient, preventing a patient to whom said formulation is orally administered from tasting said foul-tasting active, or causing the flavour of said foul-tasting at least one active or ingredient to be reduced such that it is more palatable, comprising the steps of:
(i) contacting said micro-organisms with said at least one flavouring, said at least one flavouring being capable of diffusing into the cell wall of said micro-organism without causing total lysation thereof, whereby said at least one flavouring is absorbed by said micro-organisms by diffusion across the cell wall and is retained passively within said micro-organism to produce a plurality of micro-capsules containing said at least one flavouring,
(ii) formulating said micro-capsules with said at least one active or ingredient.

9. A method of manufacture of a formulation according to claim 8, wherein said micro-organisms are additionally contacted with an at least one active or an at least one ingredient, wherein said at least one active or said at least one ingredient is foul-tasting, and is not a natural constituent of said micro-organisms, said at least one active or said at least one ingredient being encapsulated and passively retained within said micro-capsules.

10. A method of manufacture of a formulation according to claim 8, wherein said formulation additionally comprises an at least one additional flavouring.

11. A method of manufacture of a formulation according to of claim 8 wherein said at least one flavouring or said at least one additional flavouring additionally comprises ammonium glycyrrhizinate and/or polyvinylpyrrolidone.

12. A formulation according to claim 1, wherein said micro-organism is selected from the group consisting of-: fungus, bacterium, alga and protozoa.

13. A formulation according to claim 1, wherein said micro-organism is a yeast selected from the taxonomic order Endomycetales.

14. A method of manufacture according to claim 6, wherein said micro-organism is selected from the group consisting of: fungus, bacterium, alga and protozoa.

15. A method of manufacture according to claim 6, wherein said micro-organism is a yeast selected from the taxonomic order Endomycetales.
